(19) United States
(12) Patent Application Publication
(10) Pub. No.: US 2021/0008015 A1
(43) Pub. Date: Jan. 14, 2021

(54) HEPATOPROTECTENT PHENYLBUTYRATYRATE DRUG CONJUGATES, AND THEIR COMPOSITIONS PREPARATION AND METHODS OF USE THEREOF

(71) Applicant: Shreya S. Kavuru, (US)

(72) Inventors: Shreya S. Kavuru, Rumson, NJ (US); Kamalkishore Pati, Morganville, NJ (US)

(21) Appl. No.: 16/509,562

(22) Filed: Jul. 12, 2019

(51) Int. Cl. A61K 31/167; A61K 47/55; A61P 1/16; A61K 9/00; A61P 29/00

(52) U.S. Cl. CPC A61K 31/167; A61K 47/55; A61P 29/00; A61K 9/0019; A61K 9/0053; A61P 1/16

(57) ABSTRACT

In this current invention our primary aim to provide a phenylbutyrate conjugates and composition suitable for oral, or parenteral administration which overcomes the abovementioned problems. Phenylbutyrate conjugates are provided, which have a phenylbutyrate moiety covalently linked to a second drug such as Acetaminophen, or other drug molecules which induced hepatotoxicity. A compound for use in the treatment or prevention of a disease selected from hepatotoxicity, drug induced hepatotoxicity, or liver necrosis, and nonalcoholic steatohepatitis (NASH), and treat Pain, fever, cancer, inflammation, a composition for the treatment of ischemic injury or nerve damage in a human or animal, the compound having a structure selected from: **F-I** Formula and Formula **F-II* or anhydride, or pharmaceutically acceptable salt solvate thereof. It is one or more compounds or pharmaceutically acceptable salt or composition comprising a solvate of the foregoing.

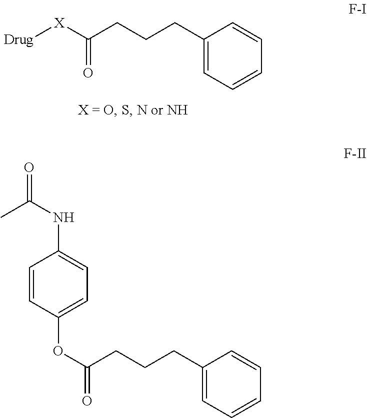

### FIELD OF INVENTION

The present invention relates to the discovery that 4-Phenylbutyricacid (PBA), Phenyl butyricacid esters and their pharmaceutically acceptable salts, when co-administered in effective amounts with a drug or other bioactive agent which typically (in the absence of the PBA compound) produces significant hepatotoxicity as a secondary indication, will substantially reduce or even eliminate such drug induced hepatotoxicity and liver necrosis and chronic alcohol consumption induced alcoholic liver steatosis, cirrhosis, and hepatocellular carcinoma. Favorable therapeutic intervention results from the use of the present invention having the effect of reducing hepatotoxicity associated with the administration of certain drugs and other bioactive agents and in certain instance of allowing the administration of higher dose of a compound which, without co administration would produce hepatotoxicity which limits or even negates the therapeutic value of the compound.

### BACKGROUND

Phenylbutyrate (PBA) is an aromatic short-chain fatty acid which is a chemical derivative of butyric acid naturally produced by colonic bacteria fermentation. At the intestinal level butyrate exerts a multitude of activities such as diminish colonel cancer, improvement of mucosal inflammation, and improvement in oxidative status. Similarly, phenylbutyrate displays potentially favorable effects on many pathologies including cancer, genetic metabolic syndromes, neuropathies, diabetes, hemoglobinopathies, and the chemical chaperone—PBA is a drug approved by the U.S. Food and Drug Administration for urea cycle disorders treatment. The mechanisms by which PBA exerts these effects are different. Some of them are connected with the regulation of gene expression, playing the role of a histone deacetylase inhibitor, while others contribute to the ability of rescuing conformational abnormalities of proteins, serving as chemical chaperone, and some are dedicated to its metabolic characteristic enabling excretion of toxic ammonia, thus acting as ammonia scavenger. Phenylbutyrate may exert variable effects depending on the cell type, thus the term “butyrate paradox” has been proposed. These data indicate a broad spectrum of beneficial effects evoked by PBA with a high potential in therapy, unfolded protein response related proteins including GRP78, GRP94, C/EBP homologous protein, phospho-eIF-2α, eIF-2α, phospho-JNK1 (p46) and phospho-JNK2/3 (p54), JNK1, IRE-1α, PERK, and sXBP-1. Endoplasmic reticulum (ER) stress is closely connected to autophagy. When cells are exposed to ER stress, cells exhibit enhanced protein degradation and form autophagosomes. From the past decade numerous studies have shown that endoplasmic reticulum (ER) stress contributes to the progression of liver disease (Ji and Kaplowitz, 2003; Malhi and Kaufman, 2011; Tan et al., 2013and Dara et al., 2011; Fernandez et al., 2013). ER stress and initiation of the unfolded protein response (UPR) is caused by accumulation of unfolded proteins in the ER, a cellular organelle that is important for the regulation of calcium homeostasis, lipid metabolism, and protein synthesis. The UPR pathway includes induction of several molecular chaperones that restore cellular homeostasis by promoting the folding or degradation of unfolded proteins; That alleviates ER stress by assisting in protein folding (Roy et al., 2015). Recently Kusuma et al reported PBA prevents murine dietary steatohepatitis caused by trans-fatty acids plus fructose by minimizing ER stress (Morinaga et al., 2015). This invention suggests that 4-PBA conjugates are potential therapeutic agents against ER stress-associated pathologic situations; drug induced hepatotoxicity and liver necrosis and chronic alcohol consumption induced alcoholic liver steatosis, cirrhosis, and hepatocellular carcinoma.

However, most widely used drug Acetaminophen (APAP) also known as paracetamol, N-(4-hydroxyphenyl)acetamide, or N-(4-hydroxyphenyl)ethanamide), is an effective and safe analgesic/antipyretic drug that is used around the world [1]. Acetaminophen is the analgesic that is widely used for the treatment of the various conditions associated with pain and fever. For example, acetaminophen is used to manage post-operative pain or trauma, and osteoarthritis, pain caused by chronic inflammatory conditions such as rheumatoid arthritis and lower back pain. Optionally, acetaminophen is used to treat pain due to nociceptive/neuropathic mixed etiologies, such as cancer or fibromyalgia. The administration of excessive Acetaminophen (APAP) induces Acetaminophen (APAP) hepatotoxicity is the most common cause of death due to acute liver failure in the developed world and is increasingly recognized as a significant public health problem (1, 2). The initial event in APAP induced hepatotoxicity is a toxic-metabolic injury leading to hepatocyte death by necrosis and apoptosis which rapidly progresses to acute liver failure. Acetaminophen (APAP) overdose induces severe oxidative stress followed by hepatocyte apoptosis/necrosis. Previous studies have indicated that endoplasmic reticulum (ER) stress is involved in the cell death process. This results in secondary activation of the innate immune response involving up-regulation of inflammatory cytokines with activation of NK, NKT cells and neutrophils, which significantly contributes to hepatotoxicity and mortality (3, 4). APAP overdose as a result of suicidal or unintentional ingestion is a critical issue [6, 7], and the incidence of APAP-induced liver injury has been increasing in recent years [8]. In the United States [2], the United Kingdom, and other countries [3-5]. The first-line therapy for APAP overdose is treatment with oral N-acetyl cysteine, which is a glutathione precursor; however, this has dose-related adverse effects and limited therapeutic efficacy [9]. Therefore, preeminent need to explore alternative therapeutic approaches against APAP-induced hepatotoxicity. The present invention an optimized acetaminophen phenylbutyrate ester composition aimed to provide a solution for above mentioned problems.

### BRIEF SUMMARY OF THE INVENTION

The present invention to provide an optimized acetaminophen phenylbutyrate ester preparation and composition suitable for oral, injectable administration which prevents or reduces hepatotoxicity problems and treat, Pain, fever, inflammation, a composition for the treatment of ischemic injury or nerve damage, pharmaceutically acceptable salt or composition comprising a solvate of the foregoing.

It is a further aim of the present invention to provide a method of manufacturing or synthesis of drug-Phenylbutyrate conjugates, or pharmaceutically acceptable salt and an improved phenyl butyrate drug conjugate composition suitable for human or animal use thereof. It is a yet further aim of the present invention to provide a method of use of an improved acetaminophen ester composition for oral, injectable administration.

One aspect of the present invention provides a compound, or a pharmaceutically acceptable salt thereof or solvate of the foregoing comprising an acetaminophen moiety and a PBA moiety.

In some embodiments, the present invention comprises a compound comprising an acetaminophen moiety, or other drugs moieties and a PBA moiety.

All publications cited herein, patents, patent applications and disclosure other references in their entirety are incorporated herein by reference. German Patent Application Publication No. 4327462 Pat

### BRIEF DESCRIPTION

Synthetic schemes for target compounds: compound can be prepared using different coupling ages is and reagents and acid, bases with protecting groups commonly used in organic synthesis.

### Synthetic Scheme for Formula-I:

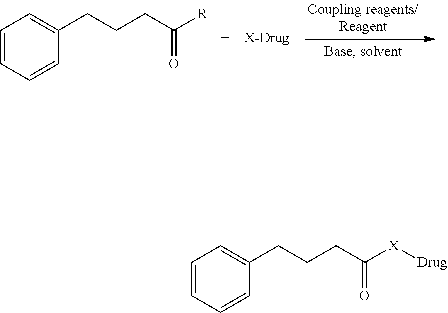

In above R can be ═OH, or O-Alkyl, O-Aryl or O-Heteroaryl, any protecting group
Drugs molecules reported in “Hepatotoxicity by

X═OH, NH, SH, SO₂ or NH₂
Drugs: The Most Common Implicated Agents”

Drug=Hepatotoxicity induceable drugs.
International Journal of Molecular Sciences reported by Einar S. Björnsson et. al., Int. J. Mol. Sci. 2016, 17, 224; doi:10.3390/ijms17020224. And all the references sited over there.

### Synthetic Scheme for Formula-II:

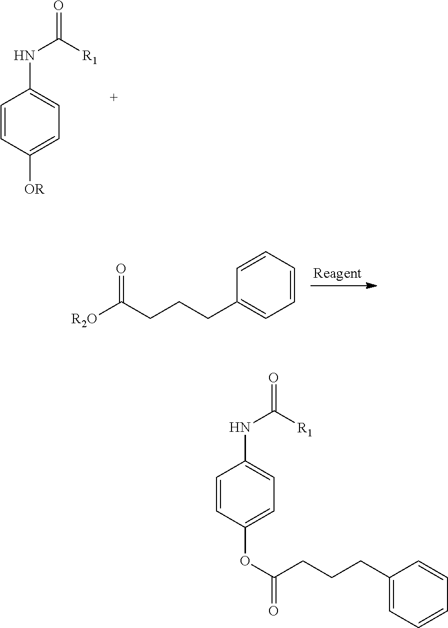 [0018] In Above R₁=Me, or Ethyl and R═H, or Protecting groups
R₂═H, Me, Ethyl, Heteroaryl, Protecting groups
Reagent can be coupling agents reported in organic synthesis; such as DCC, HATU, HOBT, DIPEA, TEA, etc.,

### General Synthetic Procedure:

Drug (1 mol) was dissolved in 50 ml Solvent, 4-Phenylbutyric acid or acid chloride or ester (1.1 mol) was added and base (0.2 mol) was added drop wise into the reaction. Coupling agent (0.2 mol) was added portion wise reaction mixture was stirred at room temperature for 5-24 hrs, the reaction was monitored using TLC, after completion of starting material Reaction mixture was poured in to 100 ml water and extracted with organic solvent 100 ml twice, the combined organic layer was washed with water twice and dried over sodium sulphate and solvent removed under reduced pressure, crude product was purified by column chromatography to obtain final product formula I and II.

### Procedure for Formula II:

N-(4-hydroxyphenyl)acetamide (1.5 gm, 0.99 mol) was dissolved in 100 ml DMF, 4-Phenylbutyric acid (16.2 gm, 0.99 mol) was added and base (Tri ethyl amine 20.8 ml, 0.148 mol) was added drop wise into the reaction. Coupling agent (HATU 45 gm, 0.1188 mol) was added portion wise reaction mixture was stirred at room temperature for 18 hr. the reaction was monitored using TLC, after completion of starting material Reaction mixture was poured in to 200 ml water and extracted with Ethyl acetate 100 ml×3 times, the combined organic layer was washed with water 200 ml×2 times and dried over sodium sulphate and solvent removed under reduced pressure, crude product was purified, final product 22 gm of N-(4-phenylbutanoyl)-N-(4-hydroxyphenyl)acetanyide was obtained.

### Formulation Preparation:

The formulations were prepared as follows: Acetaminophen phenylbutyrate conjugate was added to a mixture of 5%-10% Polysorbate 80 and water while stirring and mixing for 30 minutes to form stable micelles. The volume was then increased to 10 ml with WFI water to prepare liquid concentrate formulations. The conjugate concentrates can be diluted with any quantity of commonly used intravenous infusion solutions. The liquid concentrate can be taken as oral liquid.

Amber glass bottles were filled with the above formulations of 10 mL concentrate, with a rubber stopper and flip-off seal and subjected to stability studies under the following conditions:
In some embodiments polysorbate 80 percentage in formulations can be between 0.5% to 25%.
In some embodiments drug (APPB) concentration in formulations can be 20 mg/ml to 150 mg/ml.

ICH accelerated conditions at 40° C.±2° C./75% RH±5% RH; and

ICH room temperature conditions at 25° C.±2° C./60% RH±5% RH

Samples were analyzed to measure the acetaminophen phenylbutyrate conjugate assay, impurities. Also, physical stability of the invented formulation example physical appearance and pH drift was recorded. The stability of the concentrate is stable no assay drop or precipitation observed, and no impurities formation observed.

### In Vivo Safety and Dose Ranging Study:

A safety study was conducted in healthy 18 (male and female (M&F) Sprague Dawley (SD) rats, animals were divided into 3 groups 6 rats (3(M) & 3(F)) in each group, Group-A low dose 500 mg/kg/per day and Group-B medium dose 1000 mg/kg/per day and Group-C high dose 1500 mg/kg/per day for 5 days doses were given orally. No treatment related clinical signs were noticed throughout treatment duration and daily activities such as casting food and drinking water and moving. All group animals were survived no anomalies observed during and after treatment period animal were under observation for one week. This study clearly demonstrates this new conjugate (APPB) is safe for prolonged use at higher doses.

### In Vivo Comparative Drug Induced Hepatotoxicity Challenge Study:

An in-vivo study was performed in healthy SD rats to evaluate drug efficacy. A single dose and multi dose parallel study was conducted to evaluate the drug induced hepatotoxicity by measuring plasma AST and ALT levels. Following acclimation, overnight-fasted 6-week-old 12 SD-rats were randomly divided into two groups, 6 animals in each group (3M&3F), Group-A Test compound Acetaminophen phenylbutyrate (APPB) and Group-B reference compound Acetaminophen (AAP). Group-A animals were treated with Test compound (APPB) 800 mg/kg body weight (BW) intraperitoneal (IP) injection of APPB conjugate given and 400 mg/kg body weight (BW) reference compound (AAP) IP injection Oven to Group-B animals, and placebo IP injection given to 6 animals (3M&3F) in group-C.

randomly divided into 3 groups were given an intraperitoneal (i.p.) injection of APAP [450 mg/kg body weight (BW)]. Some of these mice were then repeatedly injected with PBA (120 mg/kg BW, i.p.) every 3 h from 0.5-3 to 12 h after

The drug induced hepatotoxicity challenge, In-life observations included cage side clinical sign observations, body weight and ALT estimation prior to start of treatment on day −1 and then reference group mice were serially euthanized by blood withdrawal after 1, 3, 6, and 12 h, after APAP injection, whereas the APPB-treated mice were euthanized by blood withdrawal after 1, 3, 6, and 12 h.

The levels of serum aspartate aminotransferase (AST) and alanine aminotransferase (ALT) activity were measured colorimetrically

### In Vivo Comparative Pain-Relieving Model Study:

12 Rabbits (M&F) (formaldehyde pain model 6 hr) were divided into 3 groups 4 rabbits in each group, Group-A Test (Acetaminophen phenylbutyrate) and Group-B reference (Acetaminophen) and Group-C Control (placebo). Group-A 4 rabbits (2M & 2F) were treated with Test 300 mg/kg orally given and 150 mg/kg; reference compound given to Group-B 4 rabbits (2M & 2F) and placebo given to group-C after 15 min each rat was dosed with 2.5% formalin in saline solution 0.1 ml was injected into middle of paw, and monitor for number of licking or scratching's happened with each animal in all groups and compared each group with other in the below table.

FIG. x. Amplitude of rubbing activity at formalin injected site
for 60 min post dose of formalin in test, reference and placebo.

| | | | Amplitude of | | | |
|---|---|---|---|---|---|---|
| | | | rubbing | | | |
| Treatment | Rabbit | No. of | activity | | | |
| (Group. No.) | Id | rubbings | (seconds) | Sum | Mean | SD |
| Group-1 | 1M | 3 | 11.84 | 36.19 | 3.62 | 9.9 |
| Test | 2M | 2 | 6.69 | | | |
| Conjugate drug | 3F | 1 | 5.02 | | | |
| APPB | 4F | 4 | 12.64 | | | |
| Group-2 | 5M | 1 | 3.1 | 16.7 | 2.78 | 6.0 |
| Reference | 6M | 2 | 6.7 | | | |
| Acetaminophen | 7F | 1 | 2.6 | | | |
| | 8F | 2 | 4.3 | | | |
| Group-3 | 9M | 2 | 9.42 | 57.42 | 5.03 | 11.41 |
| Control | 10M | 5 | 21.9 | | | |
| Placebo | 11F | 2 | 9.5 | | | |
| | 12F | 3 | 19.6 | | | |

Note:
The data was analysed statistically using unpaired t-test for comparison of test formulation 1 and reference formulation 2 with formulation 3 (placebo). The level of significance was set at p < 0.05. Data was expressed as Mean ± SD.

### In Vivo Comparative Antipyretic Effect Study:

The in vivo pharmacological study conducted the anti-pyretic activity of acetaminophen Phenylbutyrate (APPB) formulations in SD rats against yeast induced pyrexia 12 rats (M&F) (Baker's yeast induced antipyretic model 24 hr) After measuring basal T_{R} of the animals, they were injected with a pyrogenic dose of Baker's yeast (0.135 g/kg, i.p.). T_{R} changes were recorded every hour up to 4 h. Animals that showed an increase of not less than 0.5° C. in rectal temperature were selected for the experiment. Animals were randomly divided into three groups 4 rats in each group, Group-A Test (Acetaminophen phenylbutyrate) and Group-B reference (Acetaminophen) and Group-C Control (placebo). Group-A 4 rats (2M & 2F) were treated with Test 300 mg/kg orally given and 150 mg/kg reference compound given to Group-B 4 rats (2M & 2F) and placebo given to group-C. From the time baker yeast injection body temperature will rise keep monitor every 30-60 min once and record. And after treatment (time after treatment with test, reference and placebo) the body temperature was monitored rectally, for next 6 hr (0, 0.5, 1.0, 1.5, 2, 3, 4, and 6) using digital thermometer which can capture 0.1 F body temp ad recorded. The collected data was presented below table.

TABLE X
Effect of APPB-conjugate prepared formulation on yeast-
induced pyrexia at various time intervals in SD rats

| Group | 3 hours | 6 hours | 9 hours |
|---|---|---|---|
| Test Group-A | 1.21 ± 0.13 | 1.48 ± 0.08 | 1.83 ± 0.06 |
| Reference Group-B | 0.83 ± 0.08 | 1.31 ± 0.23 | 1.91 ± 0.14 |
| Placebo Group-C | 2.36 ± 0.06 | 2.57 ± 0.11 | 2.93 ± 0.08 |

Data was expressed as Mean ± SD.

shows data related to the effect of APPB-conjugate on yeast-induced pyrexia at different time intervals. Yeast injection in experimental animals caused significant rise in body temperature at the various time intervals as recorded rectally with the help of a tele-thermometer. Reference compound, a well-established antipyretic drug attenuated the rise in temperature to a significant extent at all time intervals. The APPB-conjugate significantly attenuated the rise in temperature 3 h after yeast injection After 6 and 9 h of yeast injection also the APPB-conjugate attenuated the raise in temperature in a highly significant manner in comparison to placebo control group.

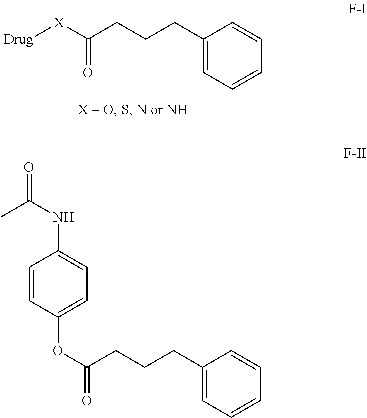

As used herein, “pharmaceutically acceptable” means biologically or no material be undesirable in other respects, for example, the material, significant undesirable biological histological effect without the bring any, or without interacting in a deleterious manner with any of the other components of it composition included, can be incorporated into a pharmaceutical composition administered to an individual (e.g., at the time of manufacture or administration). As used herein, the term “pharmaceutically acceptable carrier”, for example, an individual (e.g., human) solvents known to those skilled in the art suitable for administration to, stabilizing agents, pH adjusting agents, tonicity adjusting agents (tonicity modifier), adjuvants, binders, means such as a diluent. Combinations of two or more carriers are also contemplated in the present invention. As described herein, the component pharmaceutically acceptable carrier (s) and any additional, adapted for use in the intended route of administration of the particular dosage form (e.g., oral, parenteral) there should be sex. Such compatibility, particularly in view of the teachings provided herein will be readily recognized by those skilled in the art. Pharmaceutically acceptable carriers or excipients, contained in the pointer (Inactive Ingredient Guide) related to An effective amount” means that the composition to be administered, the condition being treated/prophylactically (e.g., the type of pain), the severity of the condition being treated or prevented, the individual's age, body size, body weight and relative health of administration route and form, the attending physician or veterinarian determined (if applicable) and in view of the teachings described herein, can vary by other factors that are well understood by those skilled in the art. An effective amount, for example, can be assessed by using one or more clinical, physiological, biochemical, histological, data from electrophysiological and/or behavioral evaluations.

In some embodiments, the individual is bovine, horse, cat, rabbit, dog, including rodents or primates, but not limited to, a mammal. In some embodiments, the mammal is a primate. In some embodiments, the primate is a human In some embodiments, the individual is a human, including adults, children, infants and premature infants. In some embodiments, the individual is a non-mammal. In some variations, the primate is a non-human primates such as chimpanzees and other apes and monkey species. In some embodiments, the mammal is a cow, horse, sheep, cattle, such as goats and swine; is rat, etc. laboratory animals including rodents such as mice and guinea pigs; rabbits, pets such as dogs and cats . In some embodiments, the individual including birds, is a non-mammal, including but not limited to. The term “individual” does not denote a particular age or gender. inactive ingredients created by satisfying the required standards of toxicological and manufacturing testing, and/or the U.S. Food and Drug Administration it is preferred that the.

1. (canceled)

2. A compound of the formula (F-II):
or a pharmaceutically acceptable salt thereof and its use to treat or cure liver diseases or of liver necrosis, and nonalcoholic steatohepatitis (NASH), and treat Pain, fever, cancer, inflammation, mentioned in above in individuals, human or animal, wherein R1 is methyl.

3. A formulation comprising a compound of claim 2 and a pharmaceutically acceptable carrier or carrier comprises composition a solvate of the foregoing.

4. A method of treating a disease or condition selected from the group treat, reduce or cure liver and disease consisting of pain, fever, inflammation, ischemic injury, and neuronal injury, comprising administering to an individual in need thereof an effective amount of a compound according to claim 1.

5. The method of claim 6, wherein the compound is administered orally or parenterally.

6. (canceled)

7. (canceled)

8. A method of treating a disease or condition selected from the group treat, reduce or cure liver and disease consisting of pain, fever, inflammation, ischemic injury, and neuronal injury, comprising administering to an individual in need thereof an effective amount of a compound according to claim 2.

9. A kit for the treatment or prevention of a disease or condition selected from the group consisting of pain, fever, inflammation, ischemic injury, and neuronal injury, comprising a compound of claim 2, or a pharmaceutically acceptable salt thereof and instructions for us.
